(19) United States
(12) Patent Application Publication
(10) Pub. No.: US 2022/0011317 A1
(43) Pub. Date: Jan. 13, 2022

(54) ISOTOPE LABELING FOR UNIVERSAL MULTIPLEXING OF METABOLITES

(71) Applicant: Saint Louis University, St. Louis, MO (US)

(72) Inventors: James Edwards, University City, MO (US); Christopher Arnatt, Ballwin, MO (US)

(73) Assignee: Saint Louis University, St. Louis, MO (US)

(21) Appl. No.: 17/293,838

(22) PCT Filed: Nov. 15, 2019
(86) PCT No.: PCT/US2019/061826

§ 371 Date: May 13, 2021

Related U.S. Application Data
(60) Provisional application No. 62/767,894, filed on Nov. 15, 2018.

(51) Int. Cl. G01N 33/60; C07C 211/09; C07C 229/12; G01N 30/72

(52) U.S. Cl. CPC G01N 33/60; C07C 211/09; G01N 2030/027; G01N 30/7233; C07B 2200/05; C07C 229/12

(57) ABSTRACT

Disclosed herein are compounds and methods for labeling and quantifying analytes using mass spectrometry.

### CROSS REFERENCE TO RELATED APPLICATIONS

This application claims priority to U.S. Provisional Application No. 62/767,894, filed Nov. 15, 2018, the content of which is incorporated herein by reference in its entirety.

### FIELD OF THE INVENTION

The present invention generally relates to compounds and methods for labeling and analyzing analytes using mass spectrometry.

### BACKGROUND OF THE INVENTION

Metabolomics offers the promise of uncovering new pathways, therapeutic targets, and biomarkers. LC-mass spectrometry (MS) based metabolomics covers a high range of metabolites but suffers from quantitation and identification difficulties due to signal response and polarity differences stemming from varying levels of hydrophobicity and charge affinity. Metabolites usually bind salts and solvents in the electrospray process resulting in irrelevant MS peaks (called degeneracy) (1) (2). This splits the signal intensity among multiple peaks and severely complicate data analysis. Metabolomics also suffers from low throughput, difficulty in identification, and dilution/poor sample loading for small samples. Current solutions include making multiple injections on multiple chromatographic methods, isotope standards (3, 4) or specific isotope tags (5-7) (8-11) (12, 13), and bioinformatic exclusion of degenerate peaks (1, 14-16). However, these solutions are not enough and this is best illustrated in the low identification rate of untargeted metabolomics and the low metabolite numbers analyzed in targeted metabolomics. Identifying metabolites from degenerate noise is difficult and determining relevant unidentified peaks to investigate further is challenging. Because much of the MS data acquired in metabolomics is discarded, tagging methods which reduce degeneracy are needed to optimize analyte signal and identification.

Advancing metabolomic technology is also critical to obtaining a holistic understanding of disease states envisioned by systems biology. Metabolomics has grown exponentially and has had a high impact on both bench science and translational arenas including: identifying new compounds relevant to pain (17, 18), predicting new modes of action of antimicrobial compounds (19, 20), revealing mechanisms in the progression of diabetes (21, 22), and evaluating heart failure (23). Current impediments in metabolomics center around the structural heterogeneity of the metabolome. Because there is no common structure or functionality like in biopolymers, a method for analyzing the whole metabolome does not exist. Rather there are a plethora of metabolomics LC-MS methods to either target a few classes of compounds (24, 25) or untargeted analysis of which <20% of the peaks are considered real metabolites (16, 26). The remaining >80% of ions are degenerate (adducts with salts, dimers, or neutral water losses) (1). In addition, identification of new potential metabolites relies on structurally informative fragmentation which is rare in current systems. For metabolomics to reach its full potential, metabolite degeneracy must be eliminated, quantitation and sensitivity improved, throughput accelerated, identification workflow strengthened and be robust enough to analyze complex samples.

Quantitative analysis of metabolites is affected by their hydrophobicity and charge affinity. Because metabolites are not biopolymers and are structurally diverse, a single method capable of loading, separating, analyzing and identifying the metabolome does not exist. Currently a large variety of platforms are used to analyze a relatively small number of analytes reproducibly. A metabolite tagging system that increases hydrophobicity and charge state would allow for pre-concentration of low abundance samples, increased signal intensity, improved chromatographic peak shape and yield useful fragmentation patterns.

There is a need for transferable tagging methods that eliminate degeneracy, increase signal intensity, and aid in identification, particularly in metabolomics.

### BRIEF SUMMARY OF THE INVENTION

Aspects of the present invention relate to compounds useful as tags/labels for analyzing analytes in a sample using mass spectrometry. In various embodiments, the compounds include those having a structure of formula (I):

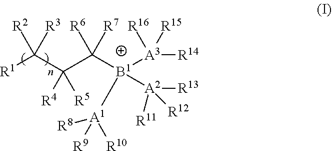

wherein:

A¹, A², and A³ are each independently ¹²C or ¹³C;

B¹ is ¹⁴N or ¹⁵N;

R¹ comprises a reactive functional group;

R², R³, R⁴, R⁵, R⁶, R⁷, R⁸, R⁹, R¹⁰, R¹¹, R¹², R¹³, R¹⁴, R¹⁵, and R¹⁶ are each independently deuterium or hydrogen; and

n is an integer from 0 to 10, from 0 to 8, from 0 to 6, from 0 to 4, from 0 to 2, from 1 to 10, from 1 to 8, from 1 to 6, from 1 to 4, or from 1 to 2.

Other aspects of the present invention also relate to various analytical methods. In some embodiments, a method of analyzing an analyte in a sample comprises reacting the analyte with a compound as described herein to label the analyte; subjecting the sample to a mass spectrometry protocol; detecting at least one charged fragment resulting from the compound that corresponds to the analyte; and analyzing the analyte based on the detection of the charged fragment.

Additional aspects of the present invention relate to various methods of preparing an analyte for analysis (e.g., quantitative analysis using mass spectrometry). In various embodiments, the methods comprise reacting the analyte with a compound as described herein (formula (I)).

Other objects and features will be in part apparent and in part pointed out hereinafter.

### BRIEF DESCRIPTION OF THE FIGURES

FIG. 1 shows problems in metabolomics and proposed solutions. Sequential tagging scheme of all functional groups (3 hr) with cationic-hydrophobic tags can clarify analyte spectra, improve quantitation, and boost sensitivity.

FIG. 2 shows two types of deuterium based isobaric universal tagging.. Tags are designed to fragment to yield a trimethyl amine neutral loss. Each tag has the exact same chemical formula, and therefore the same mass. Depending on the deuterium placement, neutral losses can be either 59, 61, 63, or 65 m/z. The cyclized product is charged and therefore detectable. The shown tags are designed to label carboxylates.

FIG. 3 shows fragmentation of quaternary amine tags on low resolution MS. Triple tagged lysine shows a +3 fixed charge. Fragmentation yields a major triple loss of trimethylamine. Both the acyl chloride tag and amine tag show fragmentation and loss of 59 as the major neutral loss. The use of these tags form five member rings instead of the proposed more stable six member ring.

FIG. 4 shows isobaric tagging of lactate for quantitation. Preliminary work of synthesizing isobaric 2-plex tags for carboxylate was performed. Each tag was reacted with lactate and mixed 1:1 followed by fragmentation of m/z 195 in a QqQ-MS. The measured ratio was 1:0.96.

FIG. 5 shows the synthesis route for universal neucode tags. Carboxylate tagging reagent (Top) and nucleophile tagging reagent (bottom). After protecting the reactive group, amines are methylated using various isotopic options of formaldehyde and CNBH₃. The deprotected carboxylic acid (bottom right) is the chlorinated to an acid chloride.

FIG. 6 show chromatographic co-elution of lactate tagged with carboxylate tag of varying deuteriums (D0 vs. D9). Deuterated carboxylate reactive tags were synthesized reacted with lactate, and analyzed for variations in retention on a RPLC-MS system. The D9 (grey) showed exact co-elution with the D0 (black).

FIGS. 7A-7C shows the results from an experiment where different concentrations of 4-formylbenzoic acid are labeled with different neucode tags of the present invention (FIG. 7A) and then detected using mass spectrometry (FIG. 7B). The peaks form the mass spectrometry plot (FIG. 7B) are used to generate a dose-response curve in FIG. 7C.

FIGS. 8A-8D show chemical reactions for preparing isotope labeled neucode tags of the present invention.

### DETAILED DESCRIPTION OF THE INVENTION

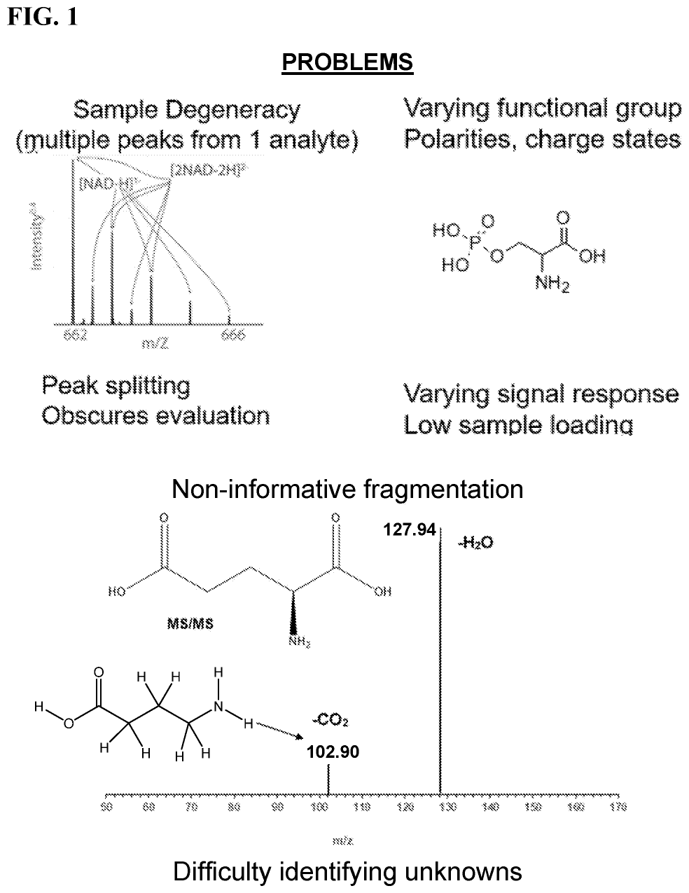

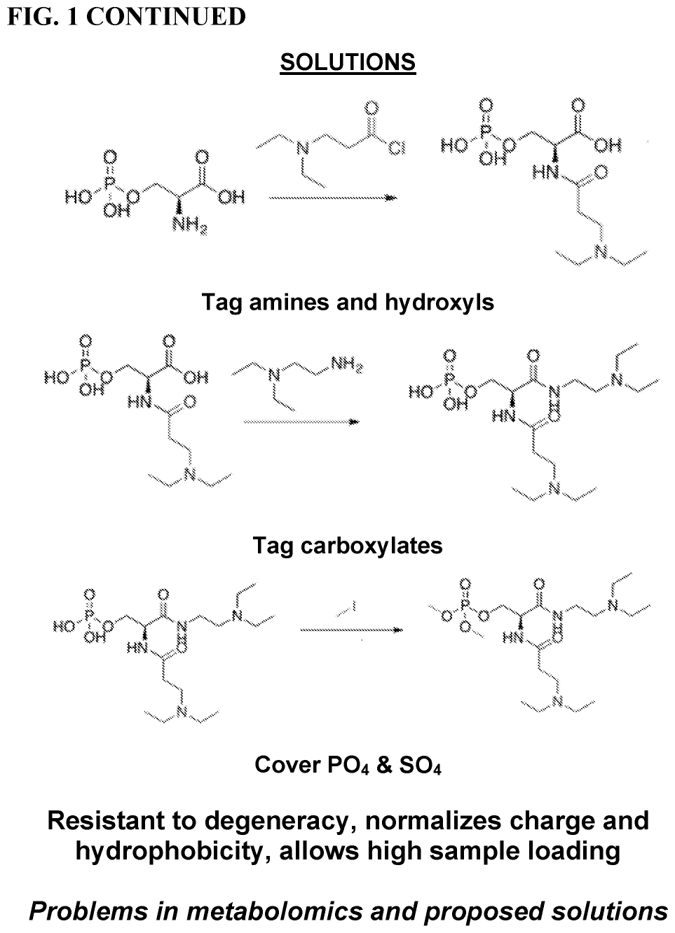

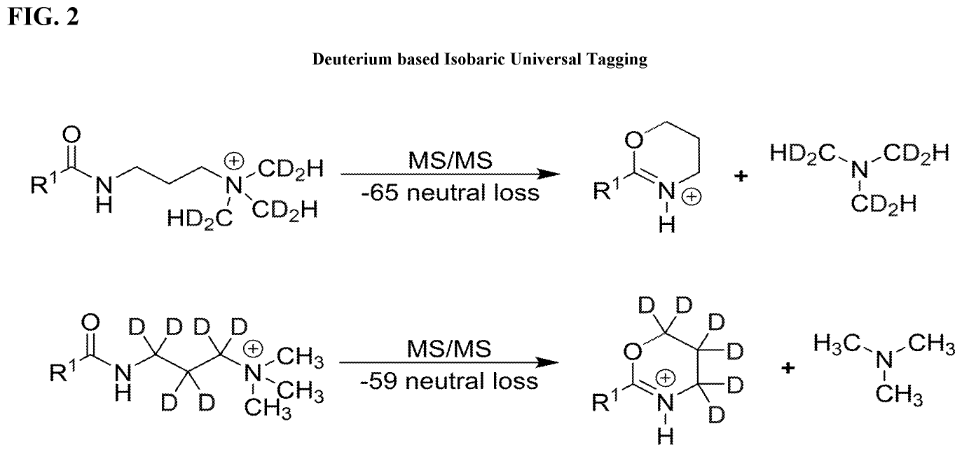

Applicants have developed a tagging technology that can improve, among other things, metabolomics throughput and data quality (FIG. 1). Analytes, such as metabolites, can be tagged in to increase both charge affinity and hydrophobicity which can: normalize signal response across metabolites, increase signal intensity, diminish degenerate signals, and allow for pre-concentration. Many analytes can be analyzed using this technique. The processes disclosed herein can enable the analysis of large analyte (e.g., metabolite) pools from precious samples using transferable protocols for both low and high resolution MS as well as the determination of the metabolome size of various samples.

Previous metabolomics work has used MS tags to react with specific functional groups which lend selectivity in analysis (32, 33). Those platforms which tag multiple functional groups do so as independent reactions (i.e. 4 different functional group tags with 4 independent samples reactions) (32-34). This breadth of analysis requires multiple LC-MS platforms/runs for each functional group tagged. The tagging scheme described herein labels the same sample sequentially, which results in multiple charges and increased hydrophobicity. Recently, the tagging method TRENDI has been used to methylate every functional group (35, 36). Drawbacks of TRENDI are that it only charges (and therefore detects) amine-containing metabolites, its reagents are highly explosive, and unshippable, which requires a synthetic chemist to generate the reagents at the time of use. Most targeted quantitative approaches use multiple platforms and focus on up to 150 compounds (6, 7, 37). Because of limited loading capability and levels of some metabolites, reproducible and targeted quantification is often ˜50 analytes. This number is expanded by using multiple LC systems (3, 4). The tagging described herein provides a universal scheme and multidimensional separations for pre-concentration and detection of all metabolites. This 2D-LC-MS platform offers a peak capacity of 1200 eliminating the need for multiple LC platforms.

Isotope tagging is used in metabolomics but has been based on specific group tagging and often only analyzing for two samples. The invention described herein uses charged tags which can selectively analyze based on functional group. Tags described herein can use differential neutral loss in a 4-plex sample. Tags for low resolution MS are 4-plex, extremely inexpensive, and functional for a diverse range of metabolites. Also provided are a set of high resolution MS tags that are based on proteomics technology of neucoding. Proteomics based neucoding uses different isotopes of lysine (¹⁵N, ¹³C, D) and up to 18-plex. The neucode system described herein uses tagging, is cost effective and can analyze up to 30-120 samples at once.

Isotope labeling is a technique in MS which improves quantitative issues of competing ionization and allows for multiplexing of samples. These techniques are often specific to a class of metabolite like primary amines or thiols. For these tags to enable quantitation, the isotopes must co-elute from the column at the same time. Tags use expensive ¹³C and ¹²C to ensure co-elution because less expensive deuterium can cause chromatographic shifts. Provided herein are methods of using deuterium-based isotope tags to substantially reduce costs for amine metabolite analyses. Specifically centering the deuterium labels around a polar center (e.g., quaternary amine) ensures chromatographic co-elution between isotopes. Isobaric tagging has been used extensively in proteomics analyses and to a lesser extent for amine based metabolomics. Isobaric tagging uses a series of labels (usually 4-12 plex) which have the same mass, but different product masses in the MS/MS fragmentation. Samples are lysed, tagged with different isobaric regents, and then mixed at equal ratios. Ratiometric quantitation is achieved in the MS/MS analyses, with each unique fragment mass corresponding to a different sample.

Neucode tagging uses isotope labels that generally have the unique fragment mass corresponding to a different sample. Neucode tagging uses isotope labels that generally have the same nominal mass but different exact mass. Because mass shifts/defects differ between ¹³C and deuterium (+1.0033 and +1.0063 Da respectively), high resolution MS can distinguish a label which has a ¹³C vs. a D.

The present invention can use deuterium based tagging to achieve universal metabolomics isotope labeling that achieves high precision at a remarkably low cost. Tags for low resolution QqQ-Ms can use a 4-plex isobaric system and the tags for high resolution MS can use a 30, 60, 90, or 120-plex neucoding system.

In general, the methods described herein can be used to tag samples with a carboxylate or nucleophile reagent and subject them to full bore or capillary RPLC-QqQ-MS. This method can be used with a low resolution targeted QqQ-MS in a targeted, product ion scan platform or with high resolution MS in a data dependent mode. With the charge state fixed to the number of tags, ¹³C spacing reveals the number of tags. This system does not suffer from competing ionization because quaternary amine tags have fixed charges and variations are accounted for through the isobaric tagging. 2D-LC is not expected to be needed.

Neucoding relies on the ability to resolve milli-dalton level differences. In proteomics, the charge state is larger than the number of isotope shifts, which requires higher resolution MS than is needed for metabolomics. Metabolomic tagging with fixed charge links the charge state with the isotopic shift (73). For instance, a singly tagged metabolite, has a +1 charge state and may have one neucode with either ¹³C₃ or D₃, which is a difference of 0.0028 Da, and 0.0028 m/z. A doubly tagged metabolite can have a +2 charge state and thus ¹³C₆ or D₆ with 0.0056 Da shift, also a difference of 0.0028 m/z. The smaller charge states on tagged metabolites vs. proteins allows for most orbitraps to be used to resolve neucode tagged metabolites.

High level multiplexing of metabolites can be achieved using neucode and fixed universal charged tagging. Based on the same general synthesis method described in Example 1 the number of isotopes around the quaternary amine can be altered using commercially available reagents. Specifically, the quaternary amine can comprise any number of deuteriums (instead of hydrogens), carbon-13 (instead of carbon-12) and nitrogen-15 (instead of nitrogen-14). In this way a library of neucode tags having detectable m/z differences can be achieved.

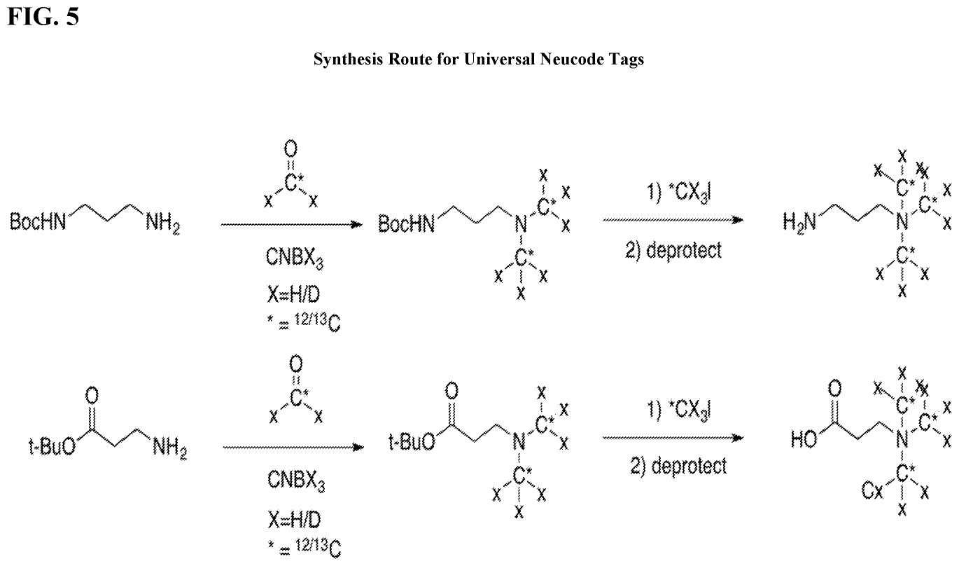

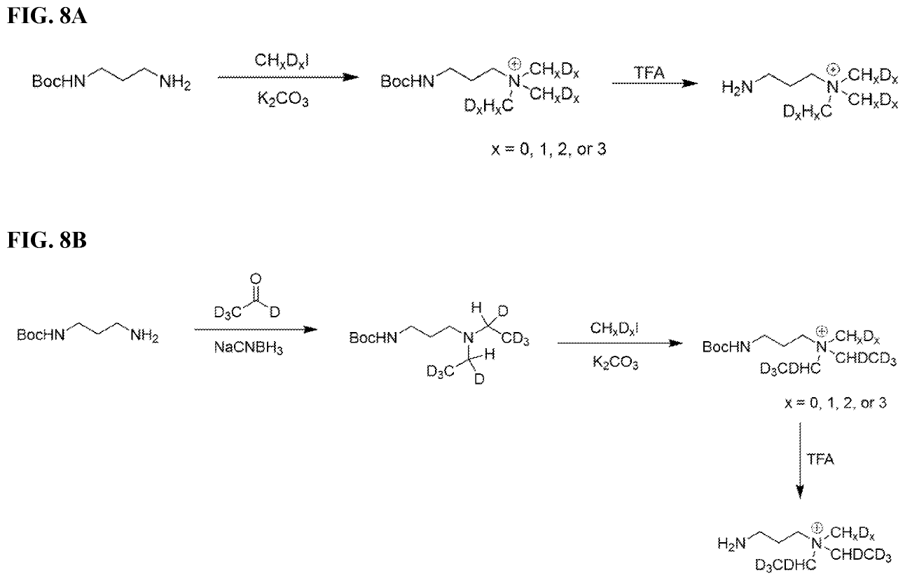

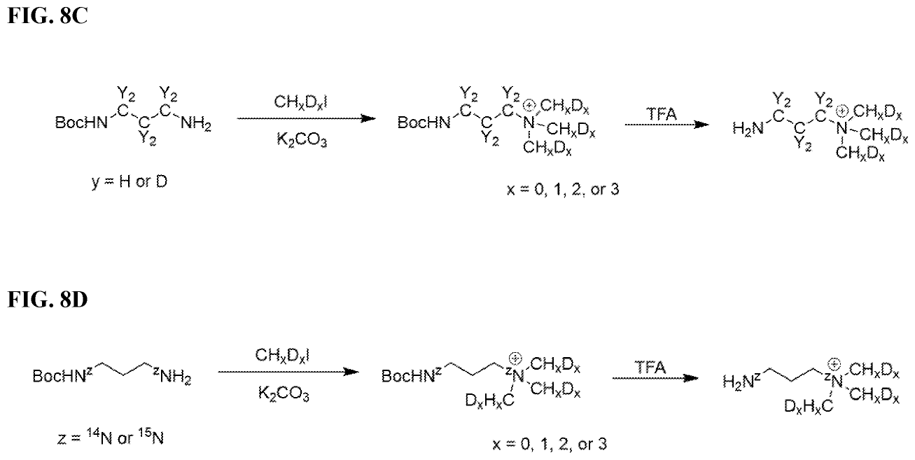

In some embodiments, a commercially available di-amino butyl chain with ¹⁵N₂ can be used. In this case, a 60-plex can be achieved while remaining very cost effective (˜$2.50/sample). To generate the acyl chloride (amine/hydroxyl reactant) tag, ¹⁵N isotopes of amino-butyric acid are commercially available to yield the 60-plex. Both tag forms can be used individually or together. Further, the methods described in FIG. 5 and FIG. 8 herein are not limited to just propyl or butyl chains. For example, one could easily envision modifying other di-amino alkyl chains could be modified in the same way (e.g., butyl, pentyl, hexyl, etc.).

This high level of multiplexing is unprecedented in metabolomics based MS. The use of commercially available reagents for synthesis allows for extremely low cost per sample. The level of MS resolution needed for these neucode tags can be determined through this protocol. In theory 30 and 60 plex are achievable because most all metabolites are below 350 m/z.

Accordingly, various aspects of the present invention relate to compounds useful as tags/labels for analyzing analytes in a sample using mass spectrometry. Other aspects of the present invention relate to various analytical methods and methods of preparing an analyte for analysis.

Compounds useful as tags/labels for analyzing analytes in a sample using mass spectrometry include those having a structure of formula (I):

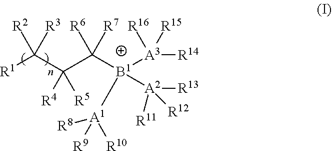

wherein:

A¹, A², and A³ are each independently ¹²C or ¹³C;

B¹ is ¹⁴N or ¹⁵N;

R¹ comprises a reactive functional group;

R², R³, R⁴, R⁵, R⁶, R⁷, R⁸, R⁹, R¹⁰, R¹¹, R¹², R¹³, R¹⁴, R¹⁵, and R¹⁶ are each independently deuterium or hydrogen; and

n is an integer from 0 to 10, from 0 to 8, from 0 to 6, from 0 to 4, from 0 to 2, from 1 to 10, from 1 to 8, from 1 to 6, from 1 to 4, or from 1 to 2.

In various embodiments, at least one substituent (i.e., atom) of formula (I) is a radioisotope. For example, in some embodiments, at least one A¹, A², A³, B¹, R², R³, R⁴, R⁵, R⁶, R⁷, R⁸, R⁹, R¹⁰, R¹¹, R¹², R¹³, R¹⁴, R¹⁵, and R¹⁶ is a radioisotope.

In certain at least one, two, or three of A¹, A², A³ is ¹³C. In these and other embodiments, at least one, two, or three of A¹, A², A³ is ¹²C.

In some embodiments, B¹ is 14N.

In various embodiments, at least one, two, three, or four of R², R³, R⁴, R⁵, R⁶, R⁷, R⁸, R⁹, R¹⁰, R¹¹, R¹², R¹³, R¹⁴, R¹⁵, and R¹⁶ is deuterium. In some embodiments, at least one, two, three or four of R⁸, R⁹, R¹⁰, R¹¹, R¹², R¹³, R¹⁴, R¹⁵, and R¹⁶ is deuterium. In certain embodiments, at least one, two, or three of R⁸, R⁹, R¹⁰ is deuterium, at least one, two, or three of R¹¹, R¹², R¹³ is deuterium; and/or at least one, two, or three of R¹⁴, R¹⁵, and R¹⁶ is deuterium. In further embodiments, R², R³, R⁴, R⁵, R⁶ and R⁷ are each deuterium.

In various embodiments, R², R³, R⁴, R⁵, R⁶ and R⁷ are each hydrogen.

As noted, R¹ comprises a reactive functional group. In some embodiments, the R¹ is a carboxyl, amido, or amino. In certain embodiments R¹ is:

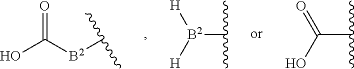

wherein B² is ¹⁴N or ¹⁵N.

In some embodiments, R¹ comprises an amido group and the compound of formula (II) forms a cyclic quaternary amine.

As noted, n can be an integer from 0 to 10, from 0 to 8, from 0 to 6, from 0 to 4, from 0 to 2, from 1 to 10, from 1 to 8, from 1 to 6, from 1 to 4, or from 1 to 2. In various embodiments, n is 1, 2, or 3.

In some embodiments, the compound having a structure of formula (I) can decay or fragment (e.g., during a mass spectrometry protocol) into compounds having a structure of formulas (II) and (III):

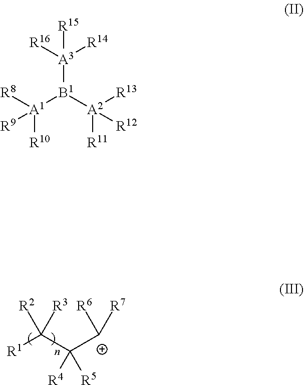

wherein A¹, A², A³, B¹, R¹, R², R³, R⁴, R⁵, R⁶, R⁷, R⁸, R⁹, R¹⁰, R¹¹, R¹², R¹³, R¹⁴, R¹⁵, and R¹⁶ are as previously defined and wherein the compound of formula (III) can be complexed or bound to an analyte.

In various embodiments, the compound has a structure of formula (Ia), (Ib), (Ic), or (Id):

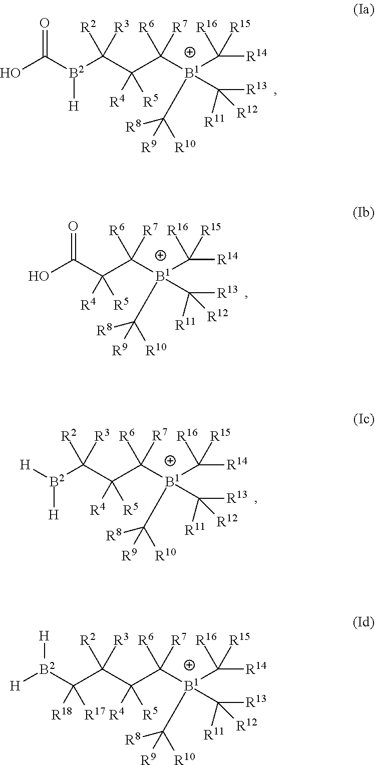

wherein:

B¹, B², R¹, R², R³, R⁴, R⁵, R⁶, R⁷, R⁸, R⁹, R¹⁰, R¹¹, R¹², R¹³, R¹⁴, R¹⁵, and R¹⁶ are as defined above; and R¹⁷ and R¹⁸ are each independently deuterium or hydrogen.

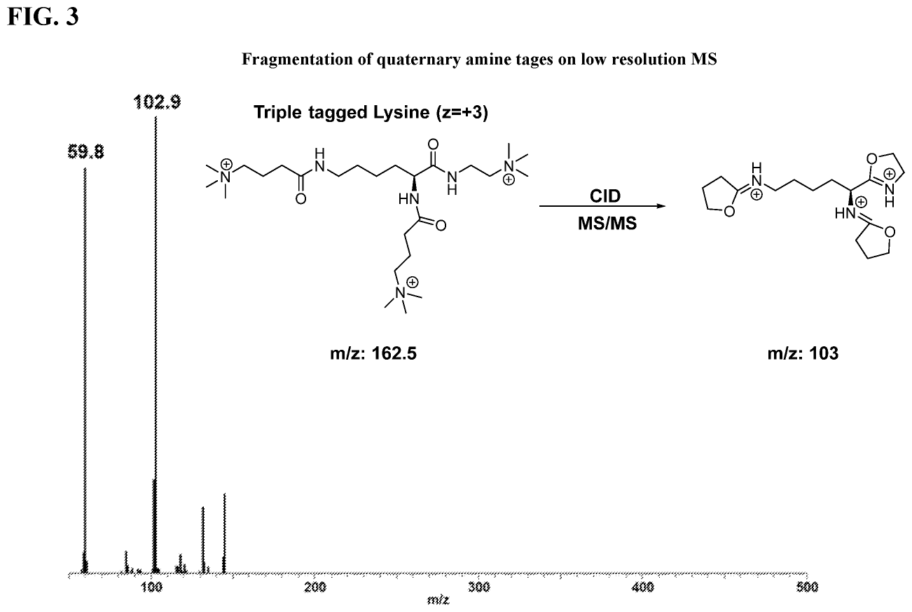

In various embodiments, isobaric tags which use only deuterium are provided. Two tags are synthesized to react with either nucleophiles (amines and hydroxyls) or carboxylates. Most isobaric tags are designed to generate a specific product ion upon fragmentation. The proof of concept for a tag that fragments with a specific neutral loss, rather than a specific ion was shown for proteomics using a sulfonium ion as a 2-plex (69-71). Here, isobaric tags are provided that are based on a quaternary amine which yield a neutral loss of trimethyl amine. FIG. 3 shows the general scheme of the system. Using commercially available deuterium starting reagents, six deuteriums can be on each of the four tags. By placing the deuterium around the nitrogen to yield a quaternary amine for one tag and the deuterium along the alkyl chain along another, the mass remains the same for each, but the fragmentation yields different product ions. By using a short chain alkyl and a quaternary amine, the loss of trimethyl amine is preferred and a stable, charged ring is generated.

As noted, aspects of the present invention relate to various methods of preparing an analyte for analysis (e.g., quantitative analysis using mass spectrometry). In various embodiments, the methods comprise reacting the analyte with the compounds as described herein (formula (I)).

Aspects of the present invention also relate to various analytical methods. In some embodiments, a method of analyzing an analyte in a sample comprise reacting the analyte with the compound as described herein to label the analyte; subjecting the sample to a mass spectrometry protocol; detecting at least one charged fragment resulting from the compound that corresponds to the analyte; and analyzing the analyte based on the detection of the charged fragment. In various embodiments, the mass spectrometry protocol comprises a triple quadrupole mass spectrometry (QqQ-MS). In certain embodiments, the mass spectrometry protocol comprises a high resolution mass spectrometry method.

In some embodiments, the method further comprises a chromatography step. In certain embodiments, the chromatography step comprises liquid chromatography or reverse phase liquid chromatography.

In various embodiments, the method comprises obtaining a plurality of samples each comprising the analyte and reacting the analyte in each sample with a compound as described herein, wherein the compound used to label the analyte is different between each sample. In some embodiments, the analytes are labeled with compounds that have the same exact mass but fragment into compounds having different m/z ratios during the mass spectrometry protocol. In other embodiments, the analytes are labeled with compounds that have the same nominal mass but different exact mass.

In various embodiments, the method comprises analyzing the analyte in the sample.

In some embodiments, the mass spectrometry protocol comprises a multiplex protocol.

In various embodiments, the analyte comprises an organic compound. In particular, the analyte can comprise an organic compound reactive with the R¹ group of the compound of formula (I). In some embodiments, the analyte comprises a protein, a nucleic acid, or a metabolite. In certain embodiments, the analyte comprises a metabolite.

In various embodiments, the sample comprises a biological specimen.

### EXAMPLES

The following non-limiting examples are provided to further illustrate the present invention.

### Example 1: Establishing Isotope Labeling for Universal Multiplexing of Metabolites

Proteomics technologies have shown proof of concept for the general approach of amine based isobaric and neucode quantitation. The following examples use isotope based tagging to achieve universal metabolomic isotope labeling that achieves high precision at a remarkably low cost. Tags for low resolution QqQ-MS use a 4-plex isobaric system (Example 2) and the tags for high resolution MS use a 30 and 60-plex neucoding system (Examples 3-5).

### Example 2: Synthesis of Universal Isobaric Tags for Targeted Analysis Using Low-Resolution QqQ-MS

In this example, the synthesis of isobaric tags (4-plex) is described for use in analyzing tagged metabolites using a low resolution mass spectrometry system (e.g., triple quadrupole mass spectrometry or QqQ-MS).

The synthesis of a four-plex set of isobaric tags was performed as described in FIG. 5 provides a scheme for the synthesis. For the carboxylate reactive tags, both 1,3-diaminopropane and 1,3-diamino(proapane-d₆) are mono-Boc protected with yields of 75% and 65% respectively. The unprotected primary amine of N-Boc-1,3-diaminopropane was then converted to three different quaternary amines with a combination of H₂/D₂ formaldehyde and NaCNBH₄ or CH₃I/CD₂HI and potassium carbonate. This system increased versatility because the isotope is inserted using two unique methylation chemistries: formaldehyde (H₂ or D₂) in a reductive amination (step one) or S_{N}2 iodomethylation (Step two). The primary amine of N-Boc-1,3-diamino(proapane-d₆) was reacted with CH₃I to yield the quaternary amine. All four subsequent compounds were then deprotected with trifluoroacetic acid yielding the primary amines with overall yields ranging from 80% to 90% after the quaternization and deprotection steps. The second type, nucleophilic reactive tags were synthesized from commercially available deuterated 4-aminobutanoic acid, and were reacted in a similar method as above to yield different quaternary amines (methyl esters formed are hydrolyzed under acidic conditions). The nucleophile reactive tags were converted into acid chlorides using Ghosez's reagent.

In preliminary studies, the nucleophile reactive tag and carboxylate reactive tag both showed strong fragmentation of a trimethylamine neutral loss (−59 m/z). FIG. 3 shows the fragmentation pattern of lysine tagged with both the carboxylate tag (blue) and the nucleophile tag (red). These data show a neutral loss of 59 as the major peak for each tag present. Lysine is tagged three times. Spectrum shows complete fragmentation in that the major product ion (102.9 m/z) is from the cleavage of all tags and that +3 charge is maintained.

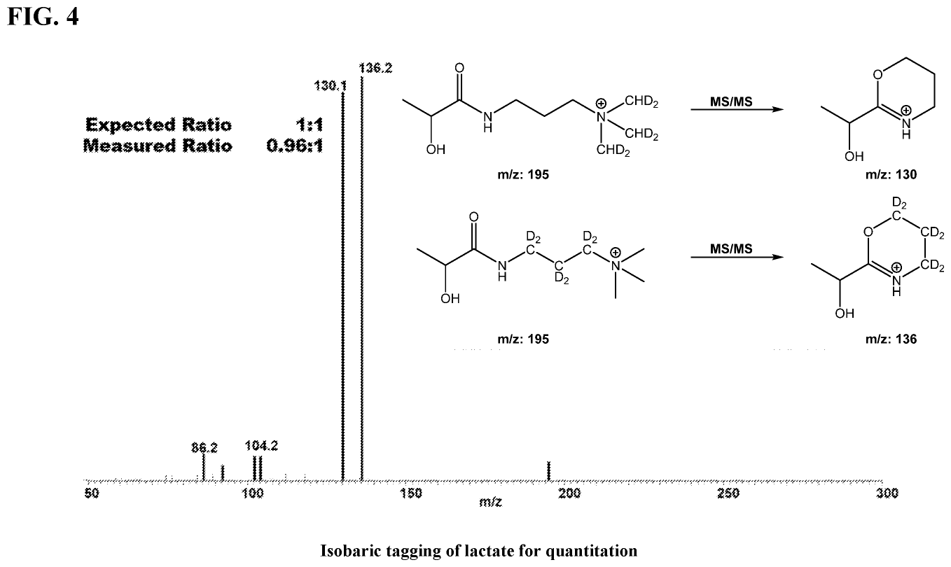

FIG. 4 shows preliminary results from using a 2-plex of this tag to quantitate tagged lactate standards. Specifically, isobaric 2-plex tags were synthesized and reacted with lactate individually. The tagged lactates were then mixed in a 1:1 ratio and fragmented using a QqQ-MS. FIG. 4 shows the resulting mass spectrometry plot. Two peaks are visible at 130 m/z and 136.2 m/z which correspond to the two different tagged lactates (each beginning with a m/z ratio of 195). The measured ratio (e.g., ratio of the peaks) was 1:0.96.

The results in this example demonstrate the feasibility and workability of isobaric tags that contain an equivalent number of hydrogens and deuteriums, but a different distribution so as to result in ionized fragments having different m/z ratios. The methods described here are useful for lower resolution mass spectrometry protocols, although they can be adapted to high resolution protocols as described above.

### Example 3: Synthesis of Universal Neucode Tags for Untargeted Analysis Using High Resolution MS

In this example, the synthesis of neucode tags (30-60-plex) is described for use in analyzing metabolites using a high resolution mass spectrometry system (e.g., RPLC-MS).

As described above, FIG. 5 shows the general synthesis route of the carboxylate (top) or nucleophile (bottom) tagging reagents. Because the addition of the first two methyl groups (formaldehyde with NaBH₃CN) is uncoupled from the last methylation, the number and variety of isotopes can be varied independently. After methylation, the protecting group is removed and the carboxylate on the nucleophile reactive tag (bottom) is converted to an acid chloride.

The wide variety of tag configurations allowed by this synthesis route can give unprecedented multiplexing capabilities. By differing the isotopes only on the quaternary amine methyls, 30 different tags with a mass difference ≥0.0028 m/z over a 12 m/z range can be achieved, and 21-plex if the m/z difference needs to be ≥0.0056 m/z. The use of quaternary amine tags reduced degeneracy and the number of potential interfering peaks within the 12 m/z range of the analyte. In some cases, tags which use ¹³C can have at least two ¹³C, to avoid interferences from the naturally occurring isotope. The use of a 12 m/z range to quantify each analyte may appear large and difficult to manage but the tagging reduces degeneracy. Adducts/neutral losses are eliminated and enhanced signal intensity of the tag eases quantitative issues.

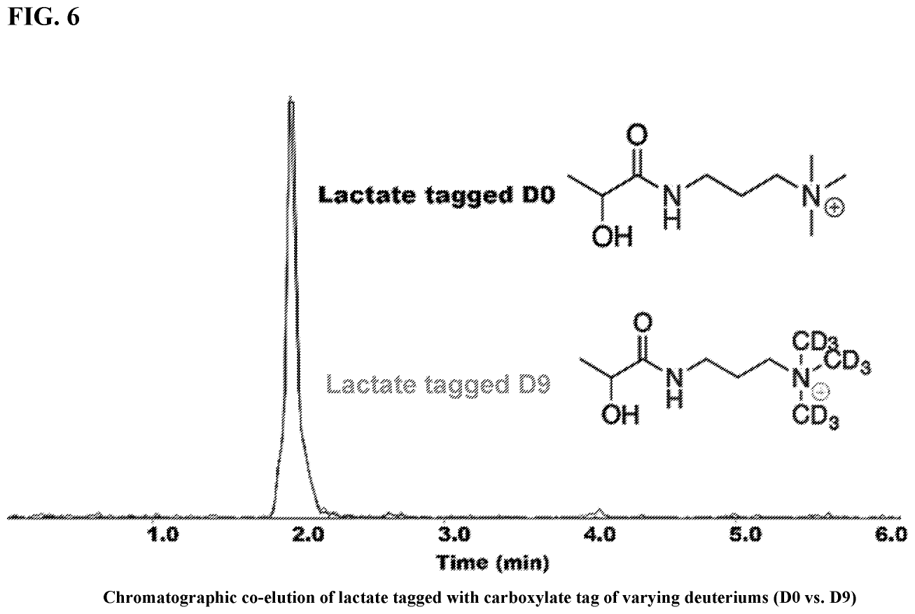

The neucode tags described here can have different masses (on account of different isotopic ratios), but should have the same polarity. Accordingly, metabolites tagged with various neucode tags can be expected to elute at the same time on a reverse-phase liquid chromatography. In support of this, lactate was tagged with deuterated carboxylate reactive tags having either 0 or 9 deuteriums and were analyzed for variations in retention time on an RPLC system. As shown in FIG. 6, both of the tags eluted at the same time, indicating no change in polarity or other properties other than the m/z ratio.

### Example 4: Formylbenzoic Acid Quantitation

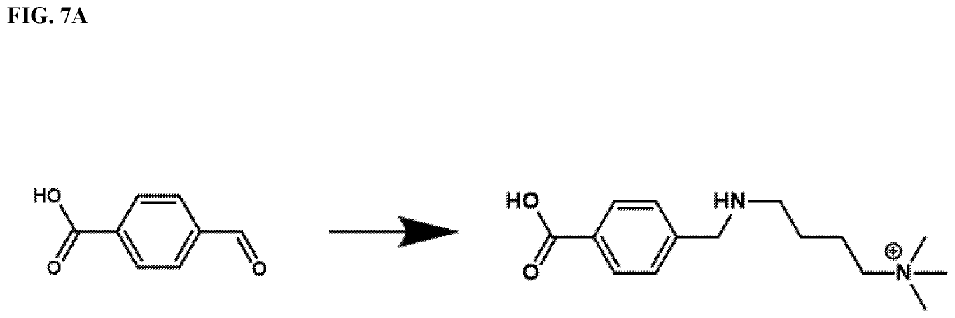

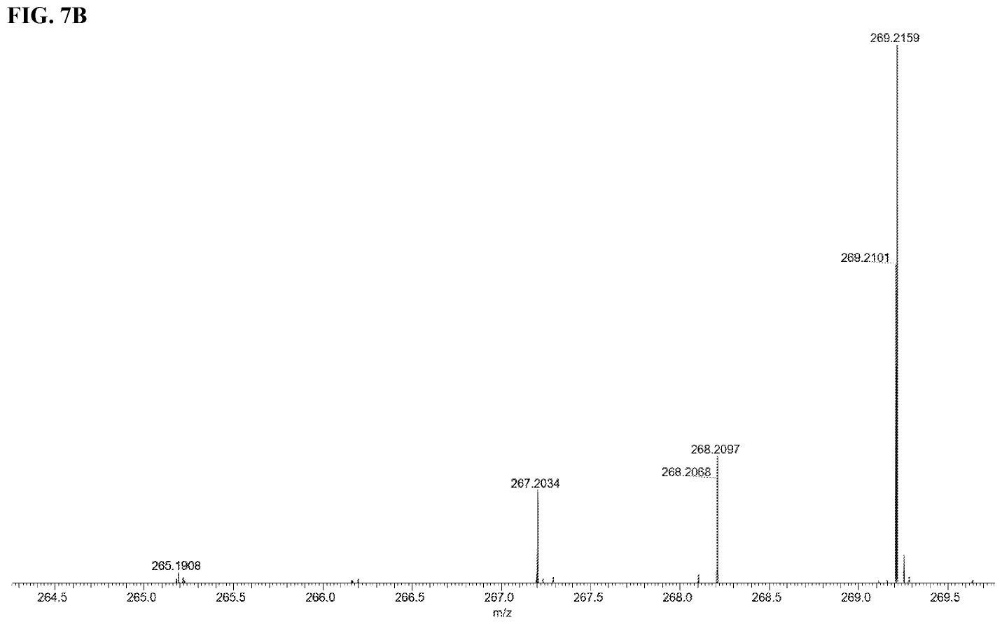

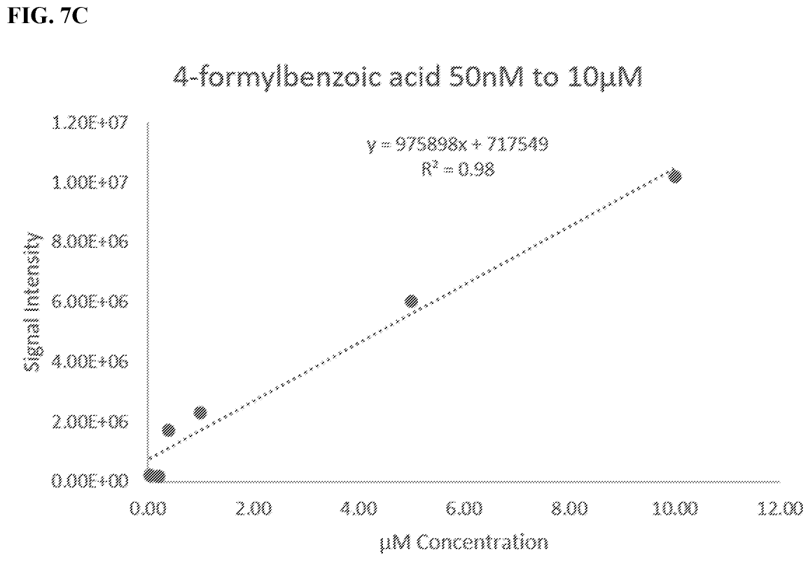

Formylbenzoic acid was reacted at varying concentration with six different neucode tag to enhance signal intensity and improve quantitation (FIG. 7A). Each concentration was tagged with a different variant of the tag as described below in the table. Samples were mixed at equal amounts and analyzed by high resolution mass spectrometry (FIG. 7B). A calibration curve was generated plotting the signal to concentration ratio and it showed a linearity of R²=0.0976 (FIG. 7C).

TABLE
4-formylbenzoic acid and neucode tag properties.

| Concentra- | Mass to charge | |
|---|---|---|
| tion (μM) | ratio (m/z) | Formula of neucode tag |
| 0.050 | 265.1911 | C₇H₁₉N₂ |
| 0.200 | 267.1978 | ¹³C₂C₅H₁₉N₂ |
| 0.400 | 267.2037 | C₇D₂H₁₇N₂ |
| 1.000 | 268.2100 | C₇D₃H₁₆N₂ |
| 5.000 | 269.2104 | ¹³C₂C₅D₂H₁₇N₂ |
| 10.000 | 269.2163 | C₇D₄H₁₅N₂ |

### Example 5: Synthesis of Isotopically Labeled Neucode Tags

The initial 30-plex using ¹³C and D around the quaternary amine (e.g., FIG. 5) can be further expanded using commercially available reactants. FIGS. 8A-8D provide reaction schemes for labeling neucode tags with different isotopes. In general, schemes are described for labeling the terminal amine in the tags described herein. For example, FIG. 8A describes how to introduce three methyl groups with the same H or D content at the same time. As an additional example, FIG. 8B describes how to introduce 2 ethyl groups with the same H or D content at the same time and 1 methyl group with varying H or D content. FIG. 8C describes how to introduce H or D into the propyl (or butyl) chain and introduce three methyl groups with the same H or D content at the same time. FIG. 8D describes how to introduce ¹⁴N or ¹⁵N into the propyl (or butyl) chain and introduce three methyl groups with the same H or D content at the same time.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compounds and methods without departing from the scope of the invention, it is intended that all matter contained in the above description and shown in the accompanying drawings shall be interpreted as illustrative and not in a limiting sense.

### REFERENCES

1. Mahieu N G, Spalding J L, Gelman S J, Patti G J. Defining and Detecting Complex Peak Relationships in Mass Spectral Data: The Mz.unity Algorithm. Anal Chem. 2016; 88(18):9037-46. Epub 2016/08/12. doi: 10.1021/acs.analchem.6b01702. PubMed PMID: 27513885.
2. Xu Y F, Lu W, Rabinowitz J D. Avoiding misannotation of in-source fragmentation products as cellular metabolites in liquid chromatography-mass spectrometry-based metabolomics. Anal Chem. 2015; 87(4):2273-81. Epub 2015/01/17. doi: 10.1021/ac504118y. PubMed PMID: 25591916; PMCID: PMC4354698.
3. Naser F J, Mahieu N G, Wang L, Spalding J L, Johnson S L, Patti G J. Two complementary reversed-phase separations for comprehensive coverage of the semipolar and nonpolar metabolome. Anal Bioanal Chem. 2018; 410(4):1287-97. Epub 2017/12/20. doi: 10.1007/s00216-017-0768-x. PubMed PMID: 29256075; PMCID: PMC5776055.
4. Ivanisevic J, Zhu Z J, Plate L, Tautenhahn R, Chen S, O'Brien P J, Johnson C H, Marletta M A, Patti G J, Siuzdak G. Toward 'omic scale metabolite profiling: a dual separation-mass spectrometry approach for coverage of lipid and central carbon metabolism. Anal Chem. 2013; 85(14):6876-84. Epub 2013/06/21. doi: 10.1021/ac401140h. PubMed PMID: 23781873; PMCID: PMC3761963.
5. Hao L, Zhong X, Greer T, Ye H, Li L. Relative quantification of amine-containing metabolites using isobaric N,N-dimethyl leucine (DiLeu) reagents via LC-ESI-MS/MS and CE-ESI-MS/MS. Analyst. 2015; 140(2):467-75. Epub 2014/11/28. doi: 10.1039/c4an01582g. PubMed PMID: 25429371; PMCID: PMC4286417.
6. Bennett B D, Yuan J, Kimball E H, Rabinowitz J D. Absolute quantitation of intracellular metabolite concentrations by an isotope ratio-based approach. Nat Protoc. 2008; 3(8):1299-311. Epub 2008/08/21. doi: 10.1038/nprot.2008.107. PubMed PMID: 18714298; PMCID: PMC2710577.
7. Lu W, Bennett B D, Rabinowitz J D. Analytical strategies for LC-MS-based targeted metabolomics. J Chromatogr B Analyt Technol Biomed Life Sci. 2008; 871(2):236-42. Epub 2008/05/27. doi: 10.1016/j.jchromb.2008.04.031. PubMed PMID: 18502704; PMCID: PMC2607197.
8. Wong J M, Malec P A, Mabrouk O S, Ro J, Dus M, Kennedy R T. Benzoyl chloride derivatization with liquid chromatography-mass spectrometry for targeted metabolomics of neurochemicals in biological samples. J Chromatogr A. 2016; 1446:78-90. Epub 2016/04/17. doi: 10.1016/j.chroma.2016.04.006. PubMed PMID: 27083258; PMCID: PMC4845038.
9. Yuan W, Anderson K W, Li S, Edwards J L. Subsecond absolute quantitation of amine metabolites using isobaric tags for discovery of pathway activation in mammalian cells. Anal Chem. 2012; 84(6):2892-9. Epub 2012/03/10. doi: 10.1021/ac203453t. PubMed PMID: 22401307.
10. Yuan W, Edwards J L, Li S. Global profiling of carbonyl metabolites with a photo-cleavable isobaric labeling affinity tag. Chem Commun (Camb). 2013; 49(94):11080-2. Epub 2013/10/22. doi: 10.1039/c3cc45956j. PubMed PMID: 24141386.
11. Yuan W, Zhang J, Li S, Edwards J L. Amine metabolomics of hyperglycemic endothelial cells using capillary LC-MS with isobaric tagging. J Proteome Res. 2011; 10(11):5242-50. Epub 2011/10/04. doi: 10.1021/pr200815c. PubMed PMID: 21961526.
12. Lane A N, Arumugam S, Lorkiewicz P K, Higashi R M, Laulhe S, Nantz M H, Moseley H N, Fan T W. Chemoselective detection and discrimination of carbonyl-containing compounds in metabolite mixtures by 1H-detected 15N nuclear magnetic resonance. Magn Reson Chem. 2015; 53(5):337-43. Epub 2015/01/24. doi: 10.1002/mrc.4199. PubMed PMID: 25616249; PMCID: PMC4409496.
13. Mattingly S J, Xu T, Nantz M H, Higashi R M, Fan T W. A Carbonyl Capture Approach for Profiling Oxidized Metabolites in Cell Extracts. Metabolomics. 2012; 8(6):989-96. Epub 2012/11/24. doi: 10.1007/s11306-011-0395-z. PubMed PMID: 23175637; PMCID: PMC3501132.
14. Crews B, Wikoff W R, Patti G J, Woo H K, Kalisiak E, Heideker J, Siuzdak G. Variability analysis of human plasma and cerebral spinal fluid reveals statistical significance of changes in mass spectrometry-based metabolomics data. Anal Chem. 2009; 81(20):8538-44. Epub 2009/09/22. doi: 10.1021/ac9014947. PubMed PMID: 19764780; PMCID: PMC3058611.
15. Mahieu N G, Huang X, Chen Y J, Patti G J. Credentialing features: a platform to benchmark and optimize untargeted metabolomic methods. Anal Chem. 2014; 86(19):9583-9. Epub 2014/08/28. doi: 10.1021/ac503092d. PubMed PMID: 25160088; PMCID: PMC4188275.
16. Mahieu N G, Patti G J. Systems-Level Annotation of a Metabolomics Data Set Reduces 25000 Features to Fewer than 1000 Unique Metabolites. Anal Chem. 2017; 89(19):10397-406. Epub 2017/09/16. doi: 10.1021/acs.analchem.7b02380. PubMed PMID: 28914531.
17. Johnson C H, Patti G J, Courade J P, Shriver L P, Hoang L T, Manchester M, Siuzdak G. Alterations in Spinal Cord Metabolism during Treatment of Neuropathic Pain. J Neuroimmune Pharmacol. 2015; 10(3):396-401. Epub 2015/08/02. doi: 10.1007/sI1481-015-9624-y. PubMed PMID: 26232265; PMCID: PMC4548716.
18. Patti G J, Yanes O, Shriver L P, Courade J P, Tautenhahn R, Manchester M, Siuzdak G. Metabolomics implicates altered sphingolipids in chronic pain of neuropathic origin. Nat Chem Biol. 2012; 8(3):232-4. Epub 2012/01/24. doi: 10.1038/nchembio.767. PubMed PMID: 22267119; PMCID: PMC3567618.
19. Zampieri M, Zimmermann M, Claassen M, Sauer U. Nontargeted Metabolomics Reveals the Multilevel Response to Antibiotic Perturbations. Cell Rep. 2017; 19(6):1214-28. Epub 2017/05/13. doi: 10.1016/j.celrep.2017.04.002. PubMed PMID: 28494870.
20. Zampieri M, Szappanos B, Buchieri M V, Trauner A, Piazza I, Picotti P, Gagneux S, Borrell S, Gicquel B, Lelievre J, Papp B, Sauer U. High-throughput metabolomic analysis predicts mode of action of uncharacterized antimicrobial compounds. Sci Transl Med. 2018; 10(429). Epub 2018/02/23. doi: 10.1126/scitranslmed.aal3973. PubMed PMID: 29467300.
21. Bain J R, Stevens R D, Wenner B R, Ilkayeva O, Muoio D M, Newgard C B. Metabolomics applied to diabetes research: moving from information to knowledge. Diabetes. 2009; 58(11):2429-43. Epub 2009/10/31. doi: 10.2337/db09-0580. PubMed PMID: 19875619; PMCID: PMC2768174.
22. Newgard C B, An J, Bain J R, Muehlbauer M J, Stevens R D, Lien L F, Haqq A M, Shah S H, Arlotto M, Slentz C A, Rochon J, Gallup D, Ilkayeva O, Wenner B R, Yancy W S, Jr., Eisenson H, Musante G, Surwit R S, Millington D S, Butler M D, Svetkey L P. A branched-chain amino acid-related metabolic signature that differentiates obese and lean humans and contributes to insulin resistance. Cell Metab. 2009; 9(4):311-26. Epub 2009/04/10. doi: 10.1016/j.cmet.2009.02.002. PubMed PMID: 19356713; PMCID: PMC3640280.
23. Deidda M, Piras C, Dessalvi C C, Locci E, Barberini L, Torri F, Ascedu F, Atzori L, Mercuro G. Metabolomic approach to profile functional and metabolic changes in heart failure. J Transl Med. 2015; 13:297. Epub 2015/09/14. doi: 10.1186/s12967-015-0661-3. PubMed PMID: 26364058; PMCID: PMC4567812.
24. Xu Y F, Letisse F, Absalan F, Lu W, Kuznetsova E, Brown G, Caudy A A, Yakunin A F, Broach J R, Rabinowitz J D. Nucleotide degradation and ribose salvage in yeast. Mol Syst Biol. 2013; 9:665. Epub 2013/05/15. doi: 10.1038/msb.2013.21. PubMed PMID: 23670538; PMCID: PMC4039369.
25. Wellen K E, Lu C, Mancuso A, Lemons J M, Ryczko M, Dennis J W, Rabinowitz J D, Coller H A, Thompson C B. The hexosamine biosynthetic pathway couples growth factor-induced glutamine uptake to glucose metabolism. Genes Dev. 2010; 24(24):2784-99. Epub 2010/11/26. doi: 10.1101/gad.1985910. PubMed PMID: 21106670; PMCID: PMC3003197.
26. Filla L A, Yuan W, Feldman E L, Li S, Edwards J L. Global metabolomic and isobaric tagging capillary liquid chromatography-tandem mass spectrometry approaches for uncovering pathway dysfunction in diabetic mouse aorta. J Proteome Res. 2014; 13(12):6121-34. Epub 2014/11/05. doi: 10.1021/pr501030e. PubMed PMID: 25368974; PMCID: PMC4261973.
27. Huang T, Armbruster M, Lee R, Hui D S, Edwards J L. Metabolomic analysis of mammalian cells and human tissue through one-pot two stage derivatizations using sheathless capillary electrophoresis-electrospray ionization-mass spectrometry. J Chromatogr A. 2018; 1567:219-25. Epub 2018/07/15. doi: 10.1016/j.chroma.2018.07.007. PubMed PMID: 30005940; PMCID: PMC6087502.
28. Huang T, Toro M, Lee R, Hui D S, Edwards J L. Multi-functional derivatization of amine, hydroxyl, and carboxylate groups for metabolomic investigations of human tissue by electrospray ionization mass spectrometry. Analyst. 2018; 143(14):3408-14. Epub 2018/06/20. doi: 10.1039/c8an00490k. PubMed PMID: 29915825; PMCID: PMC6047751.
29. Yang S, Yuan W, Yang W, Zhou J, Harlan R, Edwards J, Li S, Zhang H. Glycan analysis by isobaric aldehyde reactive tags and mass spectrometry. Anal Chem. 2013; 85(17):8188-95. Epub 2013/07/31. doi: 10.1021/ac401226d. PubMed PMID: 23895018; PMCID: PMC3785247.
30. Ryan E, Reid G E. Chemical Derivatization and Ultrahigh Resolution and Accurate Mass Spectrometry Strategies for “Shotgun” Lipidome Analysis. Acc Chem Res. 2016; 49(9):1596-604. Epub 2016/08/31. doi: 10.1021/acs.accounts.6b00030. PubMed PMID: 27575732.
31. Krusemark C J, Frey B L, Belshaw P J, Smith L M. Modifying the charge state distribution of proteins in electrospray ionization mass spectrometry by chemical derivatization. J Am Soc Mass Spectrom. 2009; 20(9):1617-25. Epub 2009/06/02. doi: 10.1016/j.jasms.2009.04.017. PubMed PMID: 19481956; PMCID: PMC2776692.
32. Carlson E E, Cravatt B F. Enrichment tags for enhanced-resolution profiling of the polar metabolome. J Am Chem Soc. 2007; 129(51):15780-2. Epub 2007/12/07. doi: 10.1021/ja0779506. PubMed PMID: 18052286.
33. Carlson E E, Cravatt B F. Chemoselective probes for metabolite enrichment and profiling. Nat Methods. 2007; 4(5):429-35. Epub 2007/04/10. doi: 10.1038/nmeth1038. PubMed PMID: 17417646.
34. Yuan B F, Zhu Q F, Guo N, Zheng S J, Wang Y L, Wang J, Xu J, Liu S J, He K, Hu T, Zheng Y W, Xu F Q, Feng Y Q. Comprehensive Profiling of Fecal Metabolome of Mice by Integrated Chemical Isotope Labeling-Mass Spectrometry Analysis. Anal Chem. 2018; 90(5):3512-20. Epub 2018/02/07. doi: 10.1021/acs.analchem.7b05355. PubMed PMID: 29406693.
35. Wasslen K V, Canez C R, Lee H, Manthorpe J M, Smith J C. Trimethylation enhancement using diazomethane (TrEnDi) II: rapid in-solution concomitant quaternization of glycerophospholipid amino groups and methylation of phosphate groups via reaction with diazomethane significantly enhances sensitivity in mass spectrometry analyses via a fixed, permanent positive charge. Anal Chem. 2014; 86(19):9523-32. Epub 2014/09/11. doi: 10.1021/ac501588y. PubMed PMID: 25208053.
36. Wasslen K V, Tan le H, Manthorpe J M, Smith J C. Trimethylation enhancement using diazomethane (TrEnDi): rapid on-column quaternization of peptide amino groups via reaction with diazomethane significantly enhances sensitivity in mass spectrometry analyses via a fixed, permanent positive charge. Anal Chem. 2014; 86(7):3291-9. Epub 2014/02/22. doi: 10.1021/ac403349c. PubMed PMID: 24555738.
37. Rabinowitz J D, Kimball E. Acidic acetonitrile for cellular metabolome extraction from Escherichia coli. Anal Chem. 2007; 79(16):6167-73. Epub 2007/07/17. doi: 10.1021/ac070470c. PubMed PMID: 17630720.
38. Rauniyar N, Yates J R, 3rd. Isobaric labeling-based relative quantification in shotgun proteomics. J Proteome Res. 2014; 13(12):5293-309. Epub 2014/10/23. doi: 10.1021/pr500880b. PubMed PMID: 25337643; PMCID: PMC4261935.
39. Overmyer K A, Tyanova S, Hebert A S, Westphall M S, Cox J, Coon J J. Multiplexed proteome analysis with neutron-encoded stable isotope labeling in cells and mice. Nat Protoc. 2018; 13(1):293-306. Epub 2018/01/13. doi: 10.1038/nprot.2017.121. PubMed PMID: 29323663; PMCID: PMC5920564.
40. Hao L, Johnson J, Lietz C B, Buchberger A, Frost D, Kao W J, Li L. Mass Defect-Based N,N-Dimethyl Leucine Labels for Quantitative Proteomics and Amine Metabolomics of Pancreatic Cancer Cells. Anal Chem. 2017; 89(2):1138-46. Epub 2017/02/15. doi: 10.1021/acs.analchem.6b03482. PubMed PMID: 28194987; PMCID: PMC5338727.
41. Bowen B P, Northen T R. Dealing with the unknown: metabolomics and metabolite atlases. J Am Soc Mass Spectrom. 2010; 21(9):1471-6. Epub 2010/05/11. doi: 10.1016/j.jasms.2010.04.003. PubMed PMID: 20452782.
42. Benton H P, Ivanisevic J, Mahieu N G, Kurczy M E, Johnson C H, Franco L, Rinehart D, Valentine E, Gowda H, Ubhi B K, Tautenhahn R, Gieschen A, Fields M W, Patti G J, Siuzdak G. Autonomous metabolomics for rapid metabolite identification in global profiling. Anal Chem. 2015; 87(2):884-91. Epub 2014/12/17. doi: 10.1021/ac5025649. PubMed PMID: 25496351; PMCID: PMC4303330.
43. Bandak B, Yi L, Roper M G. Microfluidic-enabled quantitative measurements of insulin release dynamics from single islets of Langerhans in response to 5-palmitic acid hydroxy stearic acid. Lab Chip. 2018; 18(18):2873-82. Epub 2018/08/16. doi: 10.1039/c81c00624e. PubMed PMID: 30109329; PMCID: PMC6133761.
44. McKenna J P, Dhumpa R, Mukhitov N, Roper M G, Bertram R. Glucose Oscillations Can Activate an Endogenous Oscillator in Pancreatic Islets. PLoS Comput Biol. 2016; 12(10):e1005143. Epub 2016/10/28. doi: 10.1371/journal.pcbi.1005143. PubMed PMID: 27788129; PMCID: PMC5082885.
45. Going C C, Xia Z, Williams E R. New supercharging reagents produce highly charged protein ions in native mass spectrometry. Analyst. 2015; 140(21):7184-94. Epub 2015/10/01. doi: 10.1039/c5an01710f. PubMed PMID: 26421324; PMCID: PMC4617834.
46. Cassou C A, Williams E R. Desalting protein ions in native mass spectrometry using supercharging reagents. Analyst. 2014; 139(19):4810-9. Epub 2014/08/19. doi: 10.1039/c4an01085j. PubMed PMID: 25133273; PMCID: PMC4245020.
47. Cassou C A, Sterling H J, Susa A C, Williams E R. Electrothermal supercharging in mass spectrometry and tandem mass spectrometry of native proteins. Anal Chem. 2013; 85(1):138-46. Epub 2012/12/01. doi: 10.1021/ac302256d. PubMed PMID: 23194134; PMCID: PMC3536820.
48. Ghassabian S, Wright L A, Dejager A D, Smith M T. Development and validation of a sensitive solid-phase-extraction (SPE) method using high-performance liquid chromatography/tandem mass spectrometry (LC-MS/MS) for determination of risedronate concentrations in human plasma. J Chromatogr B Analyt Technol Biomed Life Sci. 2012; 881-882:34-41. Epub 2011/12/27. doi: 10.1016/j.jchromb.2011.11.031. PubMed PMID: 22197608.
49. Martorana A M, Motta S, Sperandeo P, Mauri P, Polissi A. Differential Proteomics Based on Multidimensional Protein Identification Technology to Understand the Biogenesis of Outer Membrane of Escherichia coli. Methods Mol Biol. 2016; 1440:57-70. Epub 2016/06/18. doi: 10.1007/978-1-4939-3676-2_5. PubMed PMID: 27311664.
50. Martorana A M, Motta S, Di Silvestre D, Falchi F, Deho G, Mauri P, Sperandeo P, Polissi A. Dissecting Escherichia coli outer membrane biogenesis using differential proteomics. PLoS One. 2014; 9(6):e100941. Epub 2014/06/27. doi: 10.1371/joumal.pone.0100941. PubMed PMID: 24967819; PMCID: PMC4072712.
51. Webb K J, Xu T, Park S K, Yates J R, 3rd. Modified MuDPIT separation identified 4488 proteins in a system-wide analysis of quiescence in yeast. J Proteome Res. 2013; 12(5):2177-84. Epub 2013/04/02. doi: 10.1021/pr400027m. PubMed PMID: 23540446; PMCID: PMC3815592.
52. Smith C A, O'Maille G, Want E J, Qin C, Trauger S A, Brandon T R, Custodio D E, Abagyan R, Siuzdak G. METLIN: a metabolite mass spectral database. Ther Drug Monit. 2005; 27(6):747-51. Epub 2006/01/13. PubMed PMID: 16404815.
53. Wishart D S, Knox C, Guo A C, Eisner R, Young N, Gautam B, Hau D D, Psychogios N, Dong E, Bouatra S, Mandal R, Sinelnikov I, Xia J, Jia L, Cruz J A, Lim E, Sobsey C A, Shrivastava S, Huang P, Liu P, Fang L, Peng J, Fradette R, Cheng D, Tzur D, Clements M, Lewis A, De Souza A, Zuniga A, Dawe M, Xiong Y, Clive D, Greiner R, Nazyrova A, Shaykhutdinov R, Li L, Vogel H J, Forsythe I. HMDB: a knowledgebase for the human metabolome. Nucleic Acids Res. 2009; 37(Database issue):D₆₀₃-10. Epub 2008/10/28. doi: 10.1093/nar/gkn810. PubMed PMID: 18953024; PMCID: 2686599.
54. Kuhl C, Tautenhahn R, Bottcher C, Larson T R, Neumann S. CAMERA: an integrated strategy for compound spectra extraction and annotation of liquid chromatography/mass spectrometry data sets. Anal Chem. 2012; 84(1):283-9. Epub 2011/11/25. doi: 10.1021/ac202450g. PubMed PMID: 22111785; PMCID: 3658281.
55. Draper J, Enot D P, Parker D, Beckmann M, Snowdon S, Lin W, Zubair H. Metabolite signal identification in accurate mass metabolomics data with MZedDB, an interactive m/z annotation tool utilising predicted ionisation behaviour ‘rules’. BMC Bioinformatics. 2009; 10:227. Epub 2009/07/23. doi: 10.1186/1471-2105-10-227. PubMed PMID: 19622150; PMCID: PMC2721842.
56. Chen D, Han W, Su X, Li L, Li L. Overcoming Sample Matrix Effect in Quantitative Blood Metabolomics Using Chemical Isotope Labeling Liquid Chromatography Mass Spectrometry. Anal Chem. 2017; 89(17):9424-31. Epub 2017/08/09. doi: 10.1021/acs.analchem.7b02240. PubMed PMID: 28787119.
57. Zhao S, Dawe M, Guo K, Li L. Development of High-Performance Chemical Isotope Labeling LC-MS for Profiling the Carbonyl Submetabolome. Anal Chem. 2017; 89(12):6758-65. Epub 2017/05/16. doi: 10.1021/acs.analchem.7b01098. PubMed PMID: 28505421.
58. Mung D, Li L. Development of Chemical Isotope Labeling LC-MS for Milk Metabolomics: Comprehensive and Quantitative Profiling of the Amine/Phenol Submetabolome. Anal Chem. 2017; 89(8):4435-43. Epub 2017/03/18. doi: 10.1021/acs.analchem.6b03737. PubMed PMID: 28306241.
59. Greer T, Hao L, Nechyporenko A, Lee S, Vezina C M, Ricke W A, Marker P C, Bjorling D E, Bushman W, Li L. Custom 4-Plex DiLeu Isobaric Labels Enable Relative Quantification of Urinary Proteins in Men with Lower Urinary Tract Symptoms (LUTS). PLoS One. 2015; 10(8):e0135415. Epub 2015/08/13. doi: 10.1371/joumal.pone.0135415. PubMed PMID: 26267142; PMCID: PMC4534462.
60. Malec P A, Oteri M, Inferrera V, Cacciola F, Mondello L, Kennedy R T. Determination of amines and phenolic acids in wine with benzoyl chloride derivatization and liquid chromatography-mass spectrometry. J Chromatogr A. 2017; 1523:248-56. Epub 2017/08/05. doi: 10.1016/j.chroma.2017.07.061. PubMed PMID: 28774711.
61. Yuan W, Edwards J L. Thiol metabolomics of endothelial cells using capillary liquid chromatography mass spectrometry with isotope coded affinity tags. J Chromatogr A. 2011; 1218(18):2561-8. Epub 2011/03/23. doi: 10.1016/j.chroma.2011.02.063. PubMed PMID: 21420094.
62. Li Z, Tatlay J, Li L. Nanoflow LC-MS for High-Performance Chemical Isotope Labeling Quantitative Metabolomics. Anal Chem. 2015; 87(22):11468-74. Epub 2015/10/21. doi: 10.1021/acs.analchem.5b03209. PubMed PMID: 26482335.
63. Frost D C, Greer T, Li L. High-resolution enabled 12-plex DiLeu isobaric tags for quantitative proteomics. Anal Chem. 2015; 87(3):1646-54. Epub 2014/11/19. doi: 10.1021/ac503276z. PubMed PMID: 25405479; PMCID: PMC4318621.
64. Frost D C, Greer T, Xiang F, Liang Z, Li L. Development and characterization of novel 8-plex DiLeu isobaric labels for quantitative proteomics and peptidomics. Rapid Commun Mass Spectrom. 2015; 29(12):1115-24. Epub 2015/05/20. doi: 10.1002/rcm.7201. PubMed PMID: 25981542; PMCID: PMC4837894.
65. Lamos S M, Shortreed M R, Frey B L, Belshaw P J, Smith L M. Relative quantification of carboxylic acid metabolites by liquid chromatography-mass spectrometry using isotopic variants of cholamine. Anal Chem. 2007; 79(14):5143-9. Epub 2007/06/15. doi: 10.1021/ac062416m. PubMed PMID: 17563114; PMCID: PMC2538948.
66. Baughman J M, Rose C M, Kolumam G, Webster J D, Wilkerson E M, Merrill A E, Rhoads T W, Noubade R, Katavolos P, Lesch J, Stapleton D S, Rabaglia M E, Schueler K L, Asuncion R, Domeyer M, Zavala-Solorio J, Reich M, DeVoss J, Keller M P, Attie A D, Hebert A S, Westphall M S, Coon J J, Kirkpatrick D S, Dey A. NeuCode Proteomics Reveals Bap1 Regulation of Metabolism. Cell Rep. 2016; 16(2):583-95. Epub 2016/07/05. doi: 10.1016/j.celrep.2016.05.096. PubMed PMID: 27373151; PMCID: PMC5546211.
67. Potts G K, Voigt E A, Bailey D J, Rose C M, Westphall M S, Hebert A S, Yin J, Coon J J. Neucode Labels for Multiplexed, Absolute Protein Quantification. Anal Chem. 2016; 88(6):3295-303. Epub 2016/02/18. doi: 10.1021/acs.analchem.5b04773. PubMed PMID: 26882330; PMCID: PMC5141612.
68. Shortreed M R, Lamos S M, Frey B L, Phillips M F, Patel M, Belshaw P J, Smith L M. Ionizable isotopic labeling reagent for relative quantification of amine metabolites by mass spectrometry. Anal Chem. 2006; 78(18):6398-403. Epub 2006/09/15. doi: 10.1021/ac0607008. PubMed PMID: 16970314.
69. Lu Y, Zhou X, Stemmer P M, Reid G E. Sulfonium ion derivatization, isobaric stable isotope labeling and data dependent CID- and ETD-MS/MS for enhanced phosphopeptide quantitation, identification and phosphorylation site characterization. J Am Soc Mass Spectrom. 2012; 23(4):577-93. Epub 2011/09/29. doi: 10.1007/s13361-011-0190-0. PubMed PMID: 21952753; PMCID: PMC4228788.
70. Zhou X, Lu Y, Wang W, Borhan B, Reid G E. ‘Fixed charge’ chemical derivatization and data dependant multistage tandem mass spectrometry for mapping protein surface residue accessibility. J Am Soc Mass Spectrom. 2010; 21(8):1339-51. Epub 2010/05/11. doi: 10.1016/j.jasms.2010.03.047. PubMed PMID: 20452239.
71. Roberts K D, Reid G E. Leaving group effects on the selectivity of the gas-phase fragmentation reactions of side chain fixed-charge-containing peptide ions. J Mass Spectrom. 2007; 42(2):187-98. Epub 2006/12/13. doi: 10.1002/jms.1150. PubMed PMID: 17154347.
72. Chen Z, Wang Q, Lin L, Tang Q, Edwards J L, Li S, Liu S. Comparative evaluation of two isobaric labeling tags, DiART and iTRAQ. Anal Chem. 2012; 84(6):2908-15. Epub 2012/03/13. doi: 10.1021/ac203467q. PubMed PMID: 22404494.
73. Tayyari F, Gowda G A, Gu H, Raftery D. 15N-cholamine—a smart isotope tag for combining NMR- and MS-based metabolite profiling. Anal Chem. 2013; 85(18):8715-21. Epub 2013/08/13. doi: 10.1021/ac401712a. PubMed PMID: 23930664; PMCID: PMC3803152.
74. Abutokaikah M T, Frye J W, Tschampel J, Rabus J M, Bythell B J. Fragmentation Pathways of Lithiated Hexose Monosaccharides. J Am Soc Mass Spectrom. 2018; 29(8):1627-37. Epub 2018/05/10. doi: 10.1007/s13361-018-1973-3. PubMed PMID: 29740760.
75. Rabus J M, Abutokaikah M T, Ross R T, Bythell B J. Sodium-cationized carbohydrate gas-phase fragmentation chemistry: influence of glycosidic linkage position. Phys Chem Chem Phys. 2017; 19(37):25643-52. Epub 2017/09/15. doi: 10.1039/c7cp04738j. PubMed PMID: 28905070.
76. Bythell B J, Suhai S, Somogyi A, Paizs B. Proton-driven amide bond-cleavage pathways of gas-phase peptide ions lacking mobile protons. J Am Chem Soc. 2009; 131(39):14057-65. Epub 2009/09/15. doi: 10.1021/ja903883z. PubMed PMID: 19746933.
77. Bythell B J, Barofsky D F, Pingitore F, Polce M J, Wang P, Wesdemiotis C, Paizs B. Backbone cleavages and sequential loss of carbon monoxide and ammonia from protonated AGG: a combined tandem mass spectrometry, isotope labeling, and theoretical study. J Am Soc Mass Spectrom. 2007; 18(7):1291-303. Epub 2007/05/29. doi: 10.1016/j.jasms.2007.03.029. PubMed PMID: 17531501.
78. Johnston N R, Mitchell R K, Haythorne E, Pessoa M P, Semplici F, Ferrer J, Piemonti L, Marchetti P, Bugliani M, Bosco D, Berishvili E, Duncanson P, Watkinson M, Broichhagen J, Trauner D, Rutter G A, Hodson D J. Beta Cell Hubs Dictate Pancreatic Islet Responses to Glucose. Cell Metab. 2016; 24(3):389-401. Epub 2016/07/28. doi: 10.1016/j.cmet.2016.06.020. PubMed PMID: 27452146; PMCID: PMC5031557.
79. Nasteska D, Hodson D J. The role of beta cell heterogeneity in islet function and insulin release. J Mol Endocrinol. 2018; 61(1):R43-R60. Epub 2018/04/18. doi: 10.1530/JME-18-0011. PubMed PMID: 29661799; PMCID: PMC5976077.
80. Benninger R K P, Hodson D J. New Understanding of beta-Cell Heterogeneity and In Situ Islet Function. Diabetes. 2018; 67(4):537-47. Epub 2018/03/22. doi: 10.2337/dbi17-0040. PubMed PMID: 29559510; PMCID: PMC5860861.
81. Ng N H J, Teo A K K. Heterogeneity and cell fate flux in single human pancreatic islet cells. Cell Death Dis. 2018; 9(2):222. Epub 2018/02/16. doi: 10.1038/s41419-018-0269-7. PubMed PMID: 29445142; PMCID: PMC5833390.
82. Blodgett D M, Redick S D, Harlan D M. Surprising Heterogeneity of Pancreatic Islet Cell Subsets. Cell Syst. 2016; 3(4):330-2. Epub 2016/10/28. doi: 10.1016/j.cels.2016.10.009. PubMed PMID: 27788358.
83. Benninger R K, Hutchens T, Head W S, McCaughey M J, Zhang M, Le Marchand S J, Satin L S, Piston D W. Intrinsic islet heterogeneity and gap junction coupling determine spatiotemporal Ca(2)(+) wave dynamics. Biophys J. 2014; 107(11):2723-33. Epub 2014/12/04. doi: 10.1016/j.bpj.2014.10.048. PubMed PMID: 25468351; PMCID: PMC4255172.
84. Benninger R K, Piston D W. Cellular communication and heterogeneity in pancreatic islet insulin secretion dynamics. Trends Endocrinol Metab. 2014; 25(8):399-406. Epub 2014/04/01. doi: 10.1016/j.tem.2014.02.005. PubMed PMID: 24679927; PMCID: PMC4112137.
85. Filla R T, Schrell A M, Coulton J B, Edwards J L, Roper M G. Frequency-Modulated Continuous Flow Analysis Electrospray Ionization Mass Spectrometry (FM-CFA-ESI-MS) for Sample Multiplexing. Anal Chem. 2018; 90(4):2414-9. Epub 2018/01/23. doi: 10.1021/acs.analchem.7b04669. PubMed PMID: 29356503.

1. A compound having a structure of formula (I): 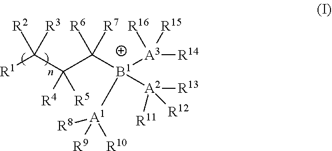
wherein:
A¹, A², and A³ are each independently ¹²C or ¹³C;
B¹ is ¹⁴N or ¹⁵N;
R¹ comprises a reactive functional group;
R², R³, R⁴, R⁵, R⁶, R⁷, R⁸, R⁹, R¹⁰, R¹¹, R¹², R¹³, R¹⁴, R¹⁵, and R¹⁶ are each independently deuterium or hydrogen; and
n is an integer from 0 to 10, from 0 to 8, from 0 to 6, from 0 to 4, from 0 to 2, from 1 to 10, from 1 to 8, from 1 to 6, from 1 to 4, or from 1 to 2.

2. The compound of claim 1 wherein at least one substituent of formula (I) is a radioisotope.

3. The compound of claim 1 wherein at least one A¹, A², A³, B¹, R², R³, R⁴, R⁵, R⁶, R⁷, R⁸, R⁹, R¹⁰, R¹¹, R¹², R¹³, R¹⁴, R¹⁵, and R¹⁶ is a radioisotope.

4. The compound of claim 1 wherein at least one, two, or three of A¹, A², A³ is ¹³C.

5. The compound of claim 1 wherein at least one, two, or three of A¹, A², A³ is ¹²C.

6. The compound of claim 1 wherein B¹ is ¹⁴N.

7. The compound of claim 1 wherein at least one, two, three, or four of R², R³, R⁴, R⁵, R⁶, R⁷, R⁸, R⁹, R¹⁰, R¹¹, R¹², R¹³, R¹⁴, R¹⁵, and R¹⁶ is deuterium.

8. (canceled)

9. The compound of claim 1 wherein at least one, two, or three of R⁸, R⁹, R¹⁰ is deuterium; at least one, two, or three of R¹¹, R¹², R¹³ is deuterium; and/or at least one, two, or three of R¹⁴, R¹⁵, and R¹⁶ is deuterium.

10. The compound of claim 1 wherein R², R³, R⁴, R⁵, R⁶ and R⁷ are each deuterium.

11. The compound of claim 1 wherein R², R³, R⁴, R⁵, R⁶ and R⁷ are each hydrogen.

12. The compound of claim 1 wherein R¹ is a carboxyl, amido, or amino.

13. The compound of claim 1 wherein R¹ is: 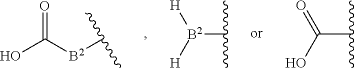
wherein B² is ¹⁴N or ¹⁵N.

14. The compound of claim 1 wherein n is 1, 2, or 3.

15. The compound of claim 1 wherein R¹ comprises an amido group and the compound of formula (I) forms a cyclic quaternary amine.

16. The compound of claim 1 wherein the compound having a structure of formula (I) can decay into compounds having a structure of formulas (II) and (III): 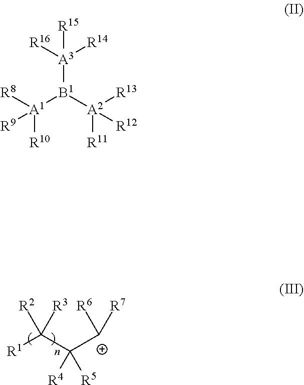
wherein A¹, A², A³, B¹, R¹, R², R³, R⁴, R⁵, R⁶, R⁷, R⁸, R⁹, R¹⁰, R¹¹, R¹², R¹³, R¹⁴, R¹⁵, and R¹⁶ are as previously defined and wherein the compound of formula (III) can be complexed or bound to an analyte.

17. The compound of claim 1 wherein the compound has a structure of formula (Ia), (Ib), (Ic), or (Id): 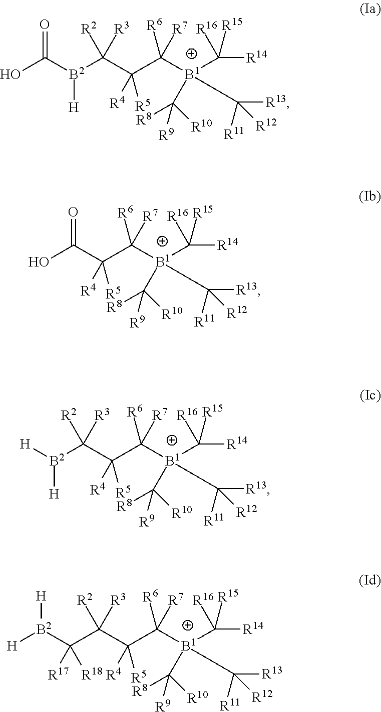
wherein:
B¹, B², R¹, R², R³, R⁴, R⁵, R⁶, R⁷, R⁸, R⁹, R¹⁰, R¹¹, R¹², R¹³, R¹⁴, R¹⁵, and R¹⁶ are as defined above; and
R¹⁷ and R¹⁸ are each independently deuterium or hydrogen.

18. A method of analyzing an analyte in a sample, the method comprising reacting the analyte with the compound of claim 1 to label the analyte; subjecting the sample to a mass spectrometry protocol; detecting at least one charged fragment resulting from the compound that corresponds to the analyte; and analyzing the analyte based on the detection of the charged fragment.

19. The method of claim 18 further comprising a chromatography step.

20-22. (canceled)

23. The method of claim 18 further comprising obtaining a plurality of samples each comprising the analyte and reacting the analyte in each sample with a compound of claim 1, wherein the compound used to label the analyte is different between each sample.

24-27. (canceled)

28. A method of preparing an analyte in a sample for analysis, the method comprising reacting the analyte with the compound of claim 1.

29-33. (canceled)
